(19) United States
(12) Patent Application Publication
(10) Pub. No.: US 2008/0200834 A1
(43) Pub. Date: Aug. 21, 2008

(54) INTRODUCER DEVICE FOR IMPROVED IMAGING

(76) Inventors: Joseph L. Mark, Indianapolis, IN (US); Zachary R. Nicoson, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/061,195

(22) Filed: Apr. 2, 2008

Related U.S. Application Data
(63) Continuation-in-part of application No. 11/550,209, filed on Oct. 17, 2006.
Application No. 11/550,209 is a continuation-in-part of application No. 11/237,110, filed on Sep. 28, 2005.

(51) Int. Cl. A61B 10/02

(52) U.S. Cl. 600/566

(57) ABSTRACT

A slotted introducer cannula includes an introducer sheath insertable into a patient's tissue. The cannula has an open distal end and an open proximal end, and an inner lumen therethrough. The introducer cannula may include at least one aperture in a side wall thereof, which allows fluid communication between the inner lumen and the area outside the cannula. The introducer cannula may also be in communication with a vacuum source, which may remove fluid near the distal end of the introducer cannula.

### CROSS-REFERENCE TO RELATED APPLICATIONS

This application is a continuation in part application of U.S. patent application Ser. No. 11/550,209, with a filing date of Oct. 17, 2006, which is a continuation in part application of U.S. patent application Ser. No. 11/237,110, with a filing date of Sep. 28, 2005, which applications are hereby incorporated herein in their entirety.

### FIELD

The present disclosure relates to the field of medical devices and more particularly to minimally invasive surgical instruments and methods.

### DESCRIPTION OF THE RELATED ART

Medical procedures have advanced to stages where less invasive or minimally invasive surgeries, diagnostic procedures and exploratory procedures have become desired and demanded by patients, physicians, and various medical industry administrators. To meet these demands, improved medical devices and instrumentation have been developed, such as cannulas or micro-cannulas, medical introducers, vacuum assisted biopsy apparatus, and other endoscopic related devices.

In the field of tissue biopsy, minimally invasive medical introduction systems have been developed that require only a single insertion point into a patient's body to remove one or more tissue samples. One such medical introduction system is configured to create a pathway to a biopsy site for precise introduction of a biopsy device, obturator devices and other medical treatments into the patient. The pathway may be defined by an introducer cannula having an open proximal end and an open distal end and defining a lumen therein. The biopsy device may incorporate a “tube-within-a-tube” design that includes an outer piercing needle having a sharpened distal end and a lateral opening that defines a tissue receiving port. An inner cutting member is slidingly received within the outer piercing needle, which serves to excise tissue that has prolapsed into the tissue receiving port. A vacuum is used to draw the excised tissue into the tissue receiving port and aspirates the excised tissue from the biopsy site once severed.

An exemplary medical introduction system is disclosed in U.S. Pat. No. 7,347,829, which is owned by the assignee of the present disclosure and is hereby incorporated herein in its entirety. Exemplary “tube-within-a-tube” biopsy devices are disclosed in U.S. Pat. Nos. 6,638,235 and 6,758,824, which are owned by the assignee of the present disclosure and are hereby incorporated herein in their entirety. Exemplary target acquisition devices are disclosed in pending U.S. patent application Ser. Nos. 11/516,277 and 11/961,505 which are owned by the assignee of the present disclosure and are hereby incorporated herein in their entirety. Among other features, the exemplary biopsy devices can be used in conjunction with Magnetic Resonance Imaging (MRI). This compatibility is due to the fact that many of the components of the devices are made of materials that do not interfere with operation of MRI apparatus or are otherwise compatible therewith. It is desirable to perform procedures such as biopsies in conjunction with MRI because it is a commonly available non-invasive visualization modality capable of defining the margins of a tumor.

While the exemplary devices have proven effective in operation, in some procedures it is may be difficult to observe devices, such as biopsy devices, obturators, etc. near a tumor or biopsy site due to fluid such as blood and air which may be located within the site. Fluid may enter the site, for example, upon removal of tissue in the biopsy site. Additionally, fluid may be displaced from within the medical introduction system upon insertion of a device within the introducer cannula. Fluid within the introducer cannula may be forced through a distal end of the lumen into a biopsy site. For these and other reasons, a system is desirable wherein fluid within or near a biopsy site can be reduced or removed, thereby improving imaging.

### BRIEF DESCRIPTION OF THE DRAWINGS

FIG. 1 is a side view of an introducer system according to an exemplary approach;

FIG. 2 is a side view of an introducer system according to another exemplary approach;

FIG. 3 is a side view of an introducer cannula according to another exemplary approach;

FIG. 4 is a side view of an introducer stylet according to an exemplary approach;

FIG. 5 is a side view of a target confirmation device according to an exemplary approach;

FIG. 6 is an elevational view illustrating an exemplary device within a treatment area;

FIG. 7 is an exemplary method of using an introducer system.

### DETAILED DESCRIPTION

Referring now to the drawings, exemplary approaches are shown in detail. Although the drawings represent some exemplary approaches, the drawings are not necessarily to scale and certain features may be exaggerated to better illustrate and explain the present disclosure. Further, the approaches set forth herein are not intended to be exhaustive or to otherwise limit or restrict the disclosure to the precise forms and configurations shown in the drawings and disclosed in the following detailed description.

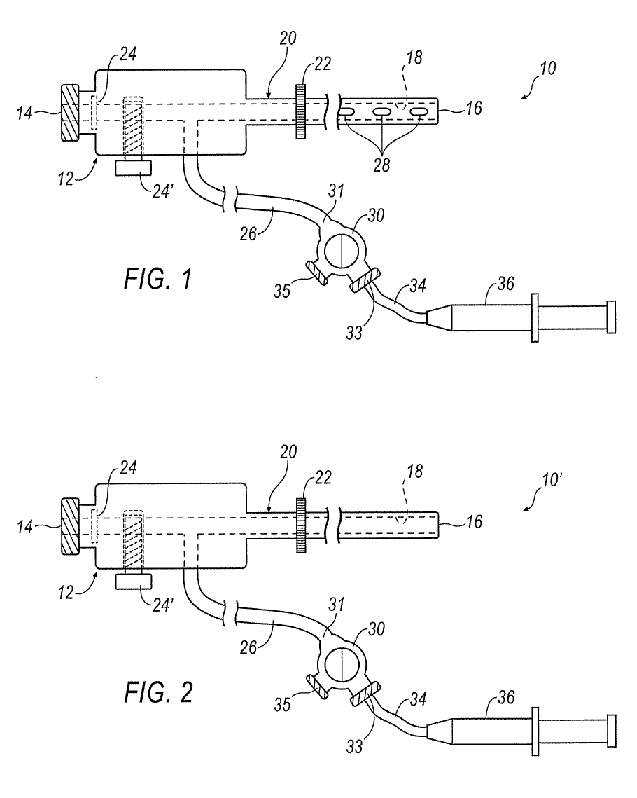

Referring to FIG. 1, an introducer system 10 is illustrated which may be used to provide a passageway to a treatment area which may be located proximate a target tissue. The introducer system 10 includes a cannula device 12 which extends from an open proximal end 14 to an open distal end 16. Cannula device 12 may be made from a medical grade resin or other MRI compatible material. In some configurations, proximal end 14 may include a luer-style fitting or other suitable configuration for interfacing, but not necessarily connecting, cannula device 12 with another device, such as a target confirmation device. Cannula device 12 may include a distal introducer sheath 20. A depth limiting member 22, such as a rubber o-ring, may be moveably disposed on introducer sheath 20 to limit the insertion depth of cannula device 12 into the patient's body.

Cannula device 12 also includes an inner lumen 18 therethrough, extending from open proximal end 14, through introducer sheath 20 to open distal end 16. Introducer sheath 20 may include at least one fluid passage slot 28 defined in an outer wall thereof, the fluid passage slot 28 allowing fluid communication between inner lumen 18 and an area external to the introducer sheath 20. For instance, introducer sheath 20 may be disposed within a passageway defined from an insertion point at which the introducer sheath 20 enters the patient's body to a treatment area proximate a target tissue within a patient's body. Fluid passage slot 28 may allow fluid to flow out of inner lumen 18 into the passageway through which introducer sheath 20 extends. Inner lumen 18 may be open to communication with a fluid conduit 26 for supplying fluids, such as saline and anesthetics, or removing fluids, such as air or blood, from the patient's body. Fluid conduit 26 may communicate with inner lumen 18 via a port in cannula device 12. In some configurations, cannula device 12 may include a haemostatic valve, depicted generally as element 24, or a manually operable valve 24′ that can be selectively closed to prevent the escape of fluid from proximal end 14. Fluid conduit 26 may also include a directional valve 30 to selectively control the supply and removal of fluid to and from inner lumen 18, respectively. Directional valve 30 may include a front port 31 in communication with fluid conduit 26, a side port 35 and a rear port 33. Fluid conduit 26 may further communicate with a vacuum source, such as syringe 36, which may cooperate with directional valve 30 through rear port 33 to selectively remove fluid from inner lumen 18. In one embodiment, the vacuum source may connect through fluid conduit 34. In the exemplary approach of FIG. 1, syringe 36 may be selectively activated to draw fluid through inner lumen 18 via outer distal opening 16. In an exemplary approach, fluid may also be drawn through at least one fluid passage slot 28.

Inner lumen 18 may be sized to receive a biopsy device, target confirmation, or similar device therein. Upon insertion if a biopsy device, target confirmation device, or similar medical device into cannula device 12, fluid may be displaced from within inner lumen 18. A portion of the fluid within inner lumen 18 may be displaced through one or more fluid passage slots 28 as the device is depressed further through inner lumen 18. Remaining fluid which is not displaced through fluid passage slots 28 may be displaced through open distal end 16. Fluid passage slots 28 may thereby reduce the amount of fluid forced into a treatment region through the open distal end 16 of cannula device 12.

FIG. 2 illustrates an exemplary introducer system 10′, which is substantially the same as introducer system 10 of FIG. 1, with the omission of the fluid passage slots 28. In the exemplary approach of FIG. 2, a vacuum source, such as syringe 36, in communication with fluid conduit 26 may draw fluid, such as air or blood, through inner lumen 18 via outer distal opening 16. The approach of FIG. 2 may allow for a focused removal of fluid from a treatment region.

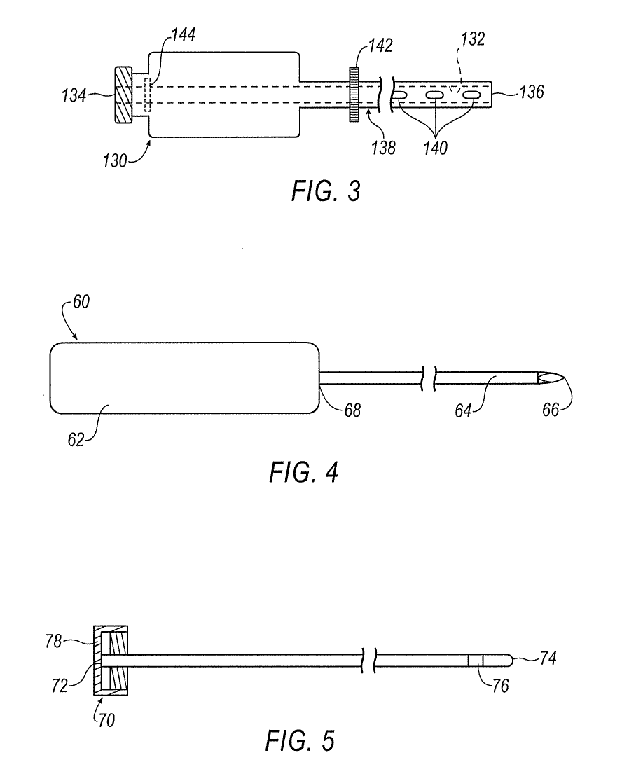

FIG. 3 illustrates an exemplary cannula device 130 having an inner lumen 132 extending from an open proximal end 134 to an open distal end 136. Cannula device 130 includes a distal introducer sheath 138. Introducer sheath 138 defines a plurality of fluid passage slots 140 in a side wall thereof, the fluid passage slots 140 allowing fluid communication between inner lumen 132 and the area external to introducer sheath 138.

FIG. 4 illustrates an exemplary introducer stylet 60 which may be useful with an introducer system such as introducer system 10 or 10′. In the exemplary approach, introducer stylet 60 includes a handle 62 and a stylet 64 having a distal end 66 and a proximal end 68 connected to handle 62. Handle 62 may be made of a medical grade resin or other MRI compatible material. Stylet 64 may also be made of an MRI compatible material, medical grade material, such as 316 stainless steel or inconel 625.

In a particular exemplary approach, a distal end 66 of stylet 64 includes a tissue piercing tip, such as a trocar tip, to facilitate penetration of stylet 64 into a patient's tissue. In addition to the trocar tip, it will be appreciated that stylet 64 may include other devices for piercing the patient's tissue, including without limitation, devices that use a laser or radio frequency (RF) to pierce the tissue. The length of stylet 64 is greater than length of cannula device 10 and 10′.

FIG. 5 illustrates an exemplary target confirmation device 70, such as an obturator. Target confirmation device 70 is an elongated member that is sized to fit within inner lumen 18 of cannula device 12. Target confirmation device 70, which may be made of a medical grade resin or other MRI compatible material, extends from a connecting end 72 to a distal end 74. Connecting end 72 may be configured with a cap 78 that abuts cannula device 12. In some configurations, cap 78 may include a luer-style fitting or other suitable feature for interfacing, but not necessarily connecting, target confirmation device 70 with cannula device 12.

Distal end 74 of target confirmation device 70 is generally rounded to facilitate entry into the patient's body. In an embodiment, a portion of target confirmation device 70 is configured with a magnetic resonance imaging (MRI) identifiable material, such as inconel 625, titanium or other material with similar magnetic characteristics. In one particular configuration, a targeting band 76 is provided in a spaced relationship from connecting end 72, as shown in FIG. 5. The distance between targeting band 76 and connecting end 72 is generally chosen to be greater than length of cannula device 10 or 10′. Targeting band 76 provides a reference point in an MR image relative to the target biopsy tissue. Other types of target confirmation devices that utilize a contrast agent as an MRI identifiable material such as Radiance, gadolinium, vitamin E, and fish oil may also be used. Examples of such may be found in co-pending U.S. patent application Ser. Nos. 11/516,277 and 11/961,505, which are owned by the assignee of the present disclosure and are hereby incorporated herein in their entireties. In another exemplary approach, the tip of target confirmation device 70 may itself be used to provide the reference point in the MR image, provided the target confirmation device material exhibits a relatively low artifact, or alternatively provides a signal void, during MR imaging. As used herein, the term “artifact” describes a material's tendency to distort an MR image. The term “signal void” describes the absence of signal, generally presented as a dark area in the MR image. A material exhibiting a relatively high artifact will render the adjacent body tissue unreadable in an MR image. Conversely, a material with a relatively low artifact will allow the material to be readily identified in the MR image and will not significantly distort the MR image of the adjacent tissue. Alternatively, a material providing a signal void will not significantly distort the MR image. In addition to materials providing a low artifact or a signal void, the target confirmation material may include any material exhibiting properties that provide for a contrasting image region against the adjacent tissue. Thus, the contrasting image region provides a reference point in an imaging modality relative to the target biopsy site. Indeed, the target confirmation material may be chosen based on performance requirements and context of use including, but not limited to, imaging modality (or modalities, if the target confirmation material may be used with multiple modalities), artifact properties, signal void properties, contrast requirements, and expected adjacent tissue properties (e.g., soft tissue, muscle tissue, brain tissue, tissue density, etc.) Further, the target confirmation material may be selected to provide intermediate levels of artifact and/or signal void.

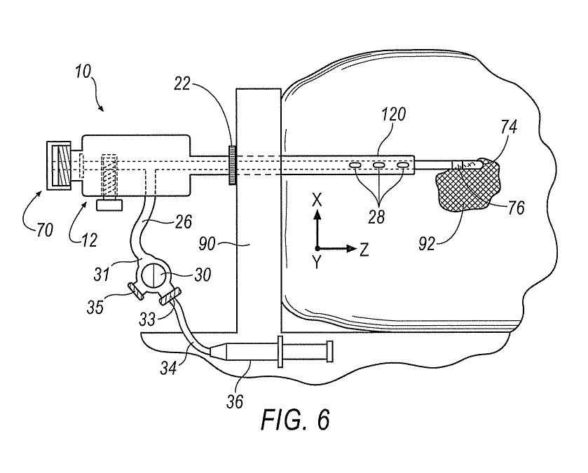

FIG. 6 illustrates an exemplary medical procedure utilizing an exemplary introducer system 10. Introducer sheath 20 may be disposed through a reference structure 90, which may be positioned adjacent to a patient to assist in locating a target tissue 92 using an imaging modality such as MRI or other suitable imaging modality.

The location of the target tissue 92 relative to reference structure 90 may be determined along one or more axis. In the illustrated embodiment, the target tissue location relative to reference structure 90 is determined along the X and Y axes; however, the target tissue location may also be determined along all three of the X, Y and Z axes. While the described method employs a reference structure 90 to locate the target tissue 92, the reference structure 90 is not necessarily required and a more “free-hand” approach may be utilized.

In an embodiment, reference structure 90 includes a support grid (not shown) having a number of holes therethrough. Each hole is sized to allow passage of introducer sheath 20 of cannula device 12. The hole through which introducer sheath 20 is ultimately inserted is determined by the location of target tissue 92 relative to reference structure 90 along the X and Y axes. The patient and reference structure 90 are viewed using a medical imaging system, such as MRI, to determine the location of the target tissue 92 relative to reference structure 90.

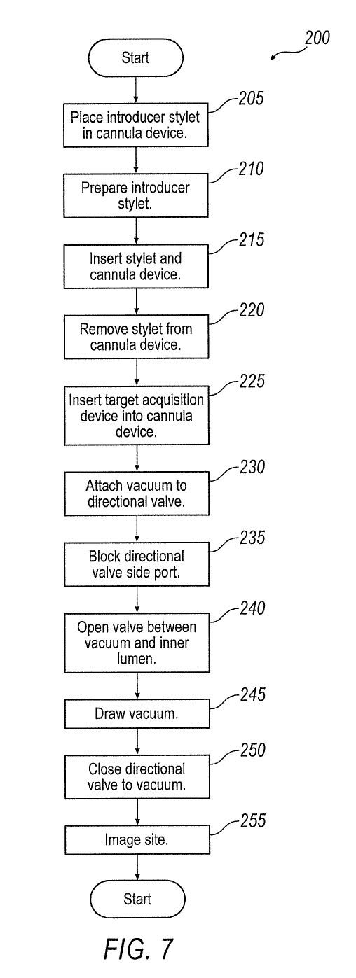

In practice, introducer sheath 20 of cannula device 12 may be inserted into a patient's body using, for example, an introducer stylet such as introducer stylet 60 illustrated in FIG. 4. Introducer stylet 60 may be inserted through cannula device 12 such that a tissue piercing tip at distal end 66 of stylet 64 extends beyond the open distal end 16 of cannula device 12. The tissue piercing tip may be inserted into the patient's tissue, creating a passageway to the target tissue 92. The stylet 64, as well as introducer sheath 20, may travel through the passageway defined by the outer piercing tip of stylet 64, into the patient's body. Once introducer sheath 20 is inserted to a desired depth, depth limiting member 22 may be moved against reference structure 90 to inhibit movement of cannula device 12 further into the patient. The introducer stylet 60 may then be removed from inner lumen 18 of cannula device 12. As introducer stylet 60 is removed from cannula device 12, fluid, such as blood and/or air, may flow into inner lumen 18, into the passageway defined by the introducer stylet 60, and/or into the treatment area around target tissue 92. A target confirmation device 70, such as an obturator, may be subsequently inserted into open proximal end 14 of cannula device 12, as illustrated in FIG. 7. As target confirmation device 70 is inserted through inner lumen 18, fluid within inner lumen 18 may be displaced. In one approach, a portion of fluid within inner lumen 18 may be forced out of the at least one fluid passage slot 28 defined within an outer wall of introducer sheath 20. Additionally, fluid which is not displaced through one or more fluid passage slot 28 may be forced out of open distal end 16. A vacuum source, such as syringe 36, is in fluid communication with inner lumen 18 of cannula device 12. Syringe 36 may be selectively operated to draw a vacuum within inner lumen 18. The vacuum source may thereby draw fluid into inner lumen 18 of introducer sheath 20 through the at least one fluid passage slot 28, and through open distal end 16. Drawing a vacuum may purge fluid from the treatment region around target tissue 92, which may allow for easier imaging of target tissue 92, and target confirmation device 70.

FIG. 7 illustrates an exemplary method 200 of using an introducer system, such as introducer system 10, together with an introducer stylet 60 and a target confirmation device 70. At step 205 an introducer stylet 60 is placed within a cannula device 12. The introducer stylet 60 and cannula device 12 are prepared for insertion into the patient at step 210. Preparation of the introducer stylet 60 and cannula device 12 may include sterilizing the introducer stylet 60 and cannula device 12 and/or spritzing the introducer stylet 60 and the introducer sheath 20 with, for example, sterile saline or lidocaine. The stylet 64 of introducer stylet 60 may be inserted into cannula device 12 such that the distal end 66 of stylet 60 extends beyond open distal end 16 of introducer sheath 20.

At step 215, the introducer stylet 60 and introducer sheath 20 of the cannula device 12 are inserted into the patient. A piercing tip at distal end 66 of stylet 64 may be depressed through a patient's tissue, toward a treatment area proximate target tissue 92. The piercing tip may form a passageway through patient's tissue through which stylet 64 and introducer sheath 20 may travel. Inserting the introducer stylet 60 and introducer sheath 20 may include observing the introducer stylet 60 and introducer sheath 20 using an imaging method, such as MRI. Further, inserting the introducer stylet 60 and introducer sheath 20 may include rotating the introducer stylet 60 and introducer sheath 20 in a continuous direction, such as clockwise or counter-clockwise, while depressing the introducer stylet 60 and introducer sheath 20 into the patient. Upon reaching a desired depth or location, a depth limiting member 22 may be used to ensure introducer sheath 20 does not extend further into the patient.

At step 220, the introducer stylet 60 is removed from the cannula device 12. Removing the introducer stylet 60 will generally cause a void near the open distal end 16 of the cannula device 12, along the passageway through which introducer stylet 60 traveled, which void may fill with fluid, such as air and/or blood.

At step 225, a target acquisition device 70 may be inserted into inner lumen 18 of cannula device 12. As target acquisition device 70 is inserted into inner lumen 18, fluid within inner lumen 18 may be displaced. A portion of fluid within inner lumen 18 may be displaced through at least one fluid passage slot 28. Additionally, a portion of fluid within inner lumen 18 may be displaced through open distal end 16 of cannula device 12, into the treatment area around target tissue 92. Fluid proximate target tissue 92 may distort an image on an imaging device, such as an MRI, which may make it difficult to view the target tissue 92, and the target acquisition device 70.

At step 230, a vacuum device, such as syringe 36, may be attached to rear port 33 of directional valve 30. Syringe 36 is generally voided prior to attachment to directional valve 30. At step 235, side port 35 of directional valve 30 is blocked.

At step 240, the directional valve is activated such that the syringe 36 is placed in fluid communication with inner lumen 18 of cannula device 12.

At step 245, a vacuum is drawn using the vacuum source. In the exemplary approach, the handle of the syringe 36 is pulled back. As the vacuum is drawn, fluid is generally drawn from the treatment area around the target tissue 92, through inner lumen 18 to the syringe 36. Fluid may be drawn through one or more fluid passage slots 28, as well as through open distal end 36 of cannula device 12. In the exemplary approach, the handle of syringe 36 may continue to be pulled until, for instance, a predetermined amount of resistance is encountered.

At step 250, the directional valve 30 is operated to cease fluid communication between the vacuum source and inner lumen 18.

Finally, at step 255, the treatment area around target tissue 92 may be imaged with an imaging device, such as MRI.

Among other features, the medical system of the present disclosure reduces fluid around a treatment area in a manner that allows improved imaging of the treatment area under MRI or other visualization modality.

The present disclosure has been particularly shown and described with reference to the foregoing approaches, which are merely illustrative of the best modes for carrying out the disclosure. It should be understood by those skilled in the art that various alternatives to the approaches described herein may be employed in practicing the disclosure without departing from the spirit and scope of the disclosure as defined in the following claims. It is intended that the following claims define the scope of the disclosure and that the method and apparatus within the scope of these claims and their equivalents be covered thereby. This description should be understood to include all novel and non-obvious combinations of elements described herein, and claims may be presented in this or a later application to any novel and non-obvious combination of these elements. Moreover, the foregoing approaches are illustrative, and no single feature or element is essential to all possible combinations that may be claimed in this or a later application.

What is claimed is:

1. A slotted introducer device, comprising:
a cannula selectively insertable into a patient's tissue, the cannula having an open distal end and an open proximal end;
wherein the cannula defines an inner lumen therein, the inner lumen extending from the open proximal end to the open distal end; and
wherein the cannula defines at least one aperture in a side wall thereof, the aperture allowing fluid communication between the inner lumen and the area outside the cannula.

2. The slotted introducer device of claim 1, wherein the cannula defines a plurality of apertures in a side wall thereof.

3. The slotted introducer device of claim 2, wherein the plurality of apertures are positioned axially about the cannula.

4. The slotted introducer device of claim 2, wherein the plurality of apertures are positioned linearly along the cannula.

5. The slotted introducer device of claim 2, wherein the plurality of apertures are spaced equidistantly about the cannula.

6. The slotted introducer device of claim 1, wherein:
the cannula further includes a fluid channel in communication with the inner lumen; and
wherein the fluid channel is in selective communication with a vacuum device.

7. The slotted introducer device of claim 6, wherein the vacuum device is operatively connected to the fluid channel through a directional valve.

8. The slotted introducer device of claim 6, wherein the vacuum device is configured to draw fluid away from a treatment area within a patient.

9. The slotted introducer device of claim 6, wherein the vacuum device is configured to selectively draw fluid through the open distal end of the introducer cannula.

10. The slotted introducer device of claim 9, wherein the vacuum device is further configured to draw fluid through the at least one aperture.

11. The slotted introducer device of claim 6, wherein the vacuum device is configured to aspirate a treatment area within a patient's body.

12. The slotted introducer device of claim 6, wherein the vacuum device is a syringe.

13. The slotted introducer device of claim 1, wherein the cannula is configured to receive a medical device therein.

14. The slotted introducer device of claim 13, wherein the medical device is one of a stylet, a biopsy device and a target confirmation device.

15. The slotted introducer device of claim 13, wherein the apertures are configured to allow fluid to be displaced from within the inner lumen to an area outside the cannula when the cannula receives a medical device therein.

16. A method, comprising:
placing a stylet in an introducer cannula, the introducer cannula including an open proximal end and an open distal end, and defining an inner lumen therein;
depressing the stylet and introducer cannula into a patient's body, thereby placing a distal end of the stylet proximate a treatment region;
removing the stylet from the introducer cannula, while leaving the introducer within the patient's body;
placing a target confirmation device within the introducer cannula;
placing a vacuum device in fluid communication with the inner lumen defined in the introducer sheath; and
selectively drawing a vacuum using the vacuum device, wherein drawing a vacuum draws fluid through the open distal end of the introducer sheath.

17. The method of claim 16, wherein selectively drawing a vacuum aspirates the treatment region.

18. The method of claim 16, wherein the introducer cannula further defines at least one aperture within a side wall thereof, and wherein selectively drawing a vacuum further draws fluid through the at least one aperture.

19. A medical introduction system, comprising:
an introducer cannula insertable into a patient's body, the introducer cannula having an open distal end and an open proximal end that defines a first length, wherein the introducer cannula defines a lumen therein;
a target confirmation device that is selectively insertable within the introducer, wherein the target confirmation device includes a distal end that extends substantially outwardly from the distal end of the introducer cannula when the target confirmation device is engaged with the introducer cannula; and
a vacuum device in fluid communication with the lumen defined within the introducer cannula, wherein the vacuum device is configured to selectively draw fluid through the open distal end of the introducer cannula.

20. The system of claim 19, wherein the introducer cannula further defines at least one aperture in a side wall thereof, the at least one aperture configured to provide fluid communication between the lumen defined within the introducer cannula and the area outside the introducer cannula.

21. The system of claim 19, wherein the outer cannula includes a fluid conduit for delivering fluid provided in communication with the lumen;
wherein the fluid conduit includes a directional valve; and
wherein the vacuum device is operatively connected to the directional valve.
